(19) United States
(12) Patent Application Publication
(10) Pub. No.: US 2006/0229276 A1
(43) Pub. Date: Oct. 12, 2006

(54) Use of glycosoaminoglycans for the prevention and treatment of sepsis

(76) Inventors: Magnus Hook, Houston, TX (US); Jorge M. Rivas, Houston, TX (US)

Correspondence Address: Benjamin Aaron Adler;ADLER & ASSOCIATES, 8011 Candle Lane, Houston, TX, 77071, US

(21) Appl. No.: 11/191,575

(22) Filed: Jul. 28, 2005

Related U.S. Application Data
(60) Provisional application No. 60/591,669, filed on Jul. 28, 2004.
(60) Provisional application No. 60/598,341, filed on Aug. 3, 2004.

(51) Int. Cl. A61K 31/737; A61K 31/727

(52) U.S. Cl. 514/54; 514/56

(57) ABSTRACT

The present invention discloses an unexpected use of glycosoaminoglycans such as low molecular weight heparin in the prevention and treatment of sepsis. Low molecular weight heparin is capable of preventing mortality and prolonging survival in a mouse model of S. aureus-induced septic death. Two other glycosaminoglycans, namely chondroitin sulfate A and dermatan sulfate were also shown to exhibit a therapeutic effect in septic mice.

### CROSS-REFERENCE TO RELATED APPLICATION

This non-provisional application claims priority of provisional application U.S. Ser. No. 60/591,669 filed Jul. 28, 2004, now abandoned, and claims priority of provisional application U.S. Ser. No. 60/598,341, filed Aug. 3, 2004, now abandoned.

### FEDERAL FUNDING LEGEND

This invention was produced in part using funds obtained through a National Institutes of Health grant (#447171-01001). Consequently, the United States government has certain rights in this invention.

### BACKGROUND OF THE INVENTION

1. Field of the Invention

The present invention relates generally to the study of sepsis. More specifically, the present invention discloses the use of glycosoaminoglycans such as low molecular weight heparin to treat sepsis and related disorders.

2. Description of the Related Art

According to U.S. Centers for Disease Control and Prevention, more than two million patients in the U.S. each year contract an infection as a result of receiving healthcare in a hospital. In 1992, cost of hospital-based infections was estimated at more than $4.5 billion in the U.S. alone. Hospital-based Staphylococcal aureus infection is an increasingly serious public health issue. In thousands of acute care hospitals in the United States, S. aureus is one of the three leading causes of hospital-based bloodstream infections, with a crude mortality rate of 25%.

While S. aureus can be contracted anywhere, it is mainly a hospital-based infection. People are natural reservoirs for S. aureus, and 30% to 50% of healthy adults are carriers of the bacteria. Infection occurs when the integrity of the skin barrier is broken, e.g., as a result of injury or surgical procedure. Patients at greatest risk are those who are immune-compromised, those whose treatment requires an invasive device such as a catheter, and those with chronic illnesses.

S. aureus infections are of special concern because of their ability to cause a number of devastating complications and their increasing resistance to current antibiotics. Serious complications from hospital Staphylococcal infections include bacteremia (blood infection), osteomyelitis (bone infection), endocarditis (infection of the inner lining of the heart and its valves), abscesses in internal organs such as the lungs, and toxic shock syndrome.

S. aureus infections have increased in the past 20 years primarily due to increase in the number of patients and increased use of invasive devices in both hospital and home care settings. Moreover, the emergence of antibiotic-resistant strains of S. aureus have also increased, thus limiting viable therapies to treat and prevent infections that can lead to a number of medical complications and death. Consequently, there is a need for improved prevention and treatment methods for such hospital-based infections. The present invention fulfills this longstanding need in the art.

### SUMMARY OF THE INVENTION

The present invention describes a novel use of glycosoaminoglycans in the prevention and treatment of sepsis and similar or related diseases and disorders. Data presented herein demonstrate an in vivo capacity of low molecular weight heparin, dermatan sulfate and chondroitin sulfate A to prevent mortality and prolong survival in a mouse model of S. aureus-induced septic death. It is unexpected that low molecular weight heparin and other glycosaminoglycans can be used to prevent and treat sepsis caused by bacteria such as S. aureus as well as related disorders and diseases.

There is no effective treatment available for staphylococcal sepsis, a malady with reported mortality rate between 30 to 70% (1). The main advantage of the present invention is that it utilizes an agent such as low molecular weight heparin that is in current clinical use and has proven to be efficacious in the treatment of other pathogenic syndromes. Moreover, low molecular weight heparin has a well documented therapeutic index and safety record.

The present invention is directed to a method of using glycosoaminoglycans to treat sepsis or a related disorder caused by bacterial infection in a human or an animal. As used herein, representative glycosoaminoglycans” include low molecular weight heparin, dermatan sulfate, chondroitin sulfate A, chondroitin sulfate C and heparan sulfate.

The invention in one embodiment gives the correlation between dose and response in S.aureus induced sepsis in mice for low molecular weight heparin (LMWH), chondroitin sulfate and dermatan sulfate.

In general, the bacterial infection is caused by gram-positive or gram-negative bacteria. The present method is particularly useful against gram-positive bacteria such as Enterococcus spp. including E. faecium, E. faecalis, E. raffinosus, E. avium, E. hirae, E. gallinarum, E. casseliflavus, E. durans, E. malodoratus, E. mundtii, E. solitarius, and E. pseudoavium; Staphylococcus spp. including S. aureus, S. epidermidis, S. hominis, S. saprophyticus, S. hemolyticus, S. capitis, S. auricularis, S. lugdenis, S. wameri, S. saccharolyticus, S. caprae, S. pasteurii, S. schleiferi, S. xylosus, S. cohnii, and S. simulans; Streptococcus spp. including S. pyogenes, S. agalactiae, S. pneumoniae, S. bovis, and viridans Streptococci.

The bacteria may be resistant to one or more antibiotics. By “antibiotic resistant” is meant any bacteria that have reduced (partially or completely) susceptibility to one or more antibiotics. Antibiotic classes to which gram-positive bacteria develop resistance include, for example, the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylcylines (e.g., doxycycline, minocycline, tetracycline, and tigecycline), the aminoglycosides (e.g., amikacin, gentamicin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid), the eveminomycins (e.g., everninonmycin), and the lipopeptides (e.g., daptomycin).

The invention also presents some aspects of the mechanism of action in dermatan sulfate protection in S. aureus-induced sepsis. The effect of dermatan sulfate on the intrinsic and extrinsic coagulation pathways was measured as a function of prothrombin time and activated partial thromboplastin time respectively. The fibrinogen and protein C levels in plasma of mice after treatment with dermatan sulphate were also evaluated.

In general, low molecular weight heparin is administered subcutaneously, but it can also be administered intraperitoneally or intravenously. In one embodiment, the low molecular weight heparin has an average molecular weight of between 1000 and 10,000 daltons. In another embodiment, the low molecular weight heparin has an average molecular weight of between 1500 and 6000 daltons. In yet another embodiment, the low molecular weight heparin has an average molecular weight of between 4000 and 5000 daltons.

Moreover, the present method described above may further comprise the step of administrating to a subject an effective amount of an agent to treat the bacterial infection. In one embodiment, such agent is an antibiotic. Uses of antibiotics against bacterial infection are readily known and available in the art. Representative antibiotics include, but are not limited to, those antibiotics listed above.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows the dose effects of low molecular weight heparin on the survival of mice infected with S. aureus strain CYL574. S. aureus strain CYL574 was grown to log phase and set via a nephelometer to a lethal concentration of 200 million cfu per mouse. Balb/c mice were infected intravenously (0.5 ml/200 million cfu) on day zero, and injected intraperitoneally with a clinical prophylaxis dose (1 mg/kg or 20 μg/mouse) or a high dose (5 mg/kg or 100 μg/mouse) of low molecular weight heparin at two hour and subsequently every twenty four hours. Mice in control group were injected intraperitoneally with PBS. Clinical appearance and weight were recorded daily. n=15 in PBS and low dose low molecular weight heparin groups; n=14 in high dose low molecular weight heparin group.

FIG. 2 shows the dose effects of low molecular weight heparin on the survival of mice infected with S. aureus strain K2. S. aureus strain K2 was grown to log phase and set via a nephelometer to a lethal concentration of 40 million cfu per mouse. Balb/c mice were infected intravenously (0.5 ml/40 million cfu) on day zero, and injected intraperitoneally with a clinical prophylaxis dose (1 mg/kg or 20 μg/mouse) or a high dose (5 mg/kg or 100 μg/mouse) of low molecular weight heparin at two hour and subsequently every twenty four hours. Mice in control group were injected intraperitoneally with PBS. Clinical appearance and weight were recorded daily. n=10 in PBS and low dose low molecular weight heparin groups; n=9 in high dose low molecular weight heparin group.

FIG. 3 shows the dose effects of low molecular weight heparin on the survival of mice infected with S. aureus strain K2. S. aureus strain K2 was grown to log phase and set via a nephelometer to a lethal concentration of 40 million cfu per mouse. Balb/c mice were infected intravenously with 40 m cfu's of S. aureus K2. Treatment groups were injected subcutaneously with increasing doses of low molecular weight heparin (5 to 40 μg of low molecular weight heparin) at two hour and subsequently every twenty four hours. Control group mice were injected subcutaneously with PBS. Clinical appearance and weight were recorded daily. n=15 in PBS and n=10 in low and high doses of low molecular weight heparin group.

FIG. 4 shows the dose effects of chondroitin sulfate (CSA) on the survival of mice infected with S. aureus strain K2. S. aureus strain K2 was grown to log phase and set via a nephelometer to a lethal concentration of 40 million cfu per mouse. Balb/c mice were infected intravenously with 40 m cfu's of S. aureus K2. Treatment groups were injected subcutaneously with increasing doses of chondroitin sulfate (50 to 2500 μg of chondroitin sulfate) at two hour and subsequently every twenty four hours. Control group mice were injected subcutaneously with PBS. Clinical appearance and weight were recorded daily. n=15 in PBS and n=10 in low dose of chondroitin sulfate and n=7 in high dose of chondroitin sulfate.

FIGS. 5A-B show the dose effect of dermatan sulfate on the survival of mice infected with a sub-lethal dose S. aureus. S. aureus K2 was grown to log phase and set via a nephelometer to an LD_{60-80%} (30 million cfu per mouse in FIG. 5A; 35 million cfu per mouse in FIG. 5B). Balb/c mice were infected intravenously (0.5 ml) on day zero. Animals were then inoculated subcutaneously with low molecular weight heparin or dermatan sulfate (20 μg/mouse) at two hour and subsequently every twenty-four hours. Control group mice were injected with PBS. Clinical appearance and weight was recorded daily. FIG. 5A: n=15 in all groups; FIG. 5B: n=20 in all groups, *p<0.025 vs. control using Fishers exact test.

FIGS. 6A-B show the dose effect of dermatan sulfate (DS) on mice infected with S. aureus strain K2. S. aureus strain K2 was grown to log phase and set via a nephelometer to a lethal concentration of 40 million cfu per mouse. Balb/c mice were infected intravenously with 40 m cfu's of S. aureus K2. Treatment groups were injected subcutaneously with increasing doses of dermatan sulfate (0.1 to 12.5 mg/kg body weight or 2 to 250 μg of dermatan sulfate) at two hour and subsequently every twenty-four hours. Control group mice were injected subcutaneously with PBS. Clinical appearance and weight were recorded daily. FIG. 6A shows the survival of mice infected with S. aureus strain K2 on treating with dermatan sulfate and FIG. 6B shows the weight loss of mice infected with S. aureus strain K2. In FIG. 6A, n=15 in PBS and n=10 for all doses of dermatan sulfate and in FIG. 6B, n=10 for both the doses of dermatan sulfate.

FIGS. 7A-B show the dose effect of dermatan sulfate (DS) on mice infected with S. aureus strain K2. S. aureus strain K2 was grown to log phase and set via a nephelometer to a lethal concentration of 40 million cfu per mouse. Balb/c mice were infected intravenously with 40 m cfu's of S. aureus K2 on day 0. Treatment groups were injected subcutaneously with increasing doses of dermatan sulfate (0.025 to 25 mg/kg body weight or 0.5 to 500 μg of dermatan sulfate) at two hour and subsequently every twenty-four hours. Control group mice were injected subcutaneously with PBS. Clinical appearance and weight were recorded daily. FIG. 7B includes a group of mice treated with a tapering dose of dermatan sulfate beginning at 16 mg/kg and decreased by half every day until the last dose on day 13 is approxmately 0.025 mg/kg body weight. In FIG. 7A, n=18 in PBS and n=10 for all doses of dermatan sulfate and in FIG. 7B, n=20 in PBS and n=12 for all doses of dermatan sulfate.

FIGS. 8A-B show the effect of dermatan sulfate (DS) on the prothrombin time (FIG. 8A) and partial thromboplastin time (FIG. 8B) of mice treated with dermatan sulfate after the onset of S.aureus-induced sepsis. K2 was grown to log phase and set via a nephelometer to a lethal concentration of 40 million cfu per mouse. Balb/c mice were infected intravenously with 40 m cfu's of S. aureus K2 on day zero. Treatment groups were injected subcutaneously with increasing doses of dermatan sulfate (50 or 500 μg dermatan sulfate per mouse) at thirty minutes and subsequently every twenty-four hours. Control group mice were injected subcutaneously with PBS or dermatan sulfate (50 or 500 μg dermatan sulfate per mouse per day). Mice were bled at 48 hours after infection via tail vein. Blood collected in 0.12 M sodium citrate in a 9:1 ratio of blood to citrate. Samples were centrifuged and plasma collected and frozen at −20° C. until use. Plasmas were diluted 1:3 in Owrens buffer for analysis. Prothrombin time (PT) and partial thromboplastin time (PTT) were determined utilizing an XM coagulometer according to manufacturer's instructions.

FIG. 9 shows the Fibrinogen levels of mice treated with dermatan sulfate (DS) after the onset of S. aureus-induced sepsis. K2 was grown to log phase and set via a nephelometer to a lethal concentration of 40 million cfu per mouse. Balb/c mice were infected intravenously with 40 m cfu's of S. aureus K2 on day zero. Treatment groups were injected subcutaneously with increasing doses of dermatan sulfate (50 or 500 μg dermatan sulfate per mouse) at thirty minutes and subsequently every twenty-four hours. Control group mice were injected subcutaneously with PBS or dermatan sulfate (50 or 500 μg dermatan sulfate per mouse per day). Mice were bled at 48 hours after infection via tail vein. Blood collected in 0.12 M sodium citrate in a 9:1 ratio of blood to citrate. Samples were centrifuged and plasma collected and frozen at −20° C. until use. Plasmas were diluted 1:9 in Owrens buffer for analysis. Fibrinogen levels were determined utilizing control standards as measured by an XM coagulometer according to manufacturer's instructions.

FIG. 10 shows the protein C levels of mice treated with dermatan sulfate (DS) after the onset of S. aureus-induced sepsis. K2 was grown to log phase and set via a nephelometer to a lethal concentration of 40 million cfu per mouse. Balb/c mice were infected intravenously with 40 million cfu's of S. aureus K2 on day zero. Treatment groups were injected subcutaneously with increasing doses of dermatan sulfate (50 or 500 μg dermatan sulfate per mouse) at thirty minutes and subsequently every twenty four hours. Control group mice were injected subcutaneously with PBS or dermatan sulfate (50 or 500 μg dermatan sulfate per mouse per day). Mice were bled at 48 hours after infection via tail vein. Blood collected in 0.12 M sodium citrate in a 9:1 ratio of blood to citrate. Samples were centrifuged and plasma collected and frozen at −20° C. until use. Plasmas were diluted 1:9 in Owrens buffer for analysis. Protein C levels were as percent levels based on standards and its ability to prolong partial thromboplastin time. Clotting assay was evaluated using an XM coagulometer according to manufacturer's instructions.

### DETAILED DESCRIPTION OF THE INVENTION

Standard heparin, a sulphated polysaccharide having an average molecular weight of 12,000-15,000 daltons, is isolated from bovine, ovine and porcine intestinal mucous membranes. Heparin is clinically used for the prevention and treatment of thromboembolic disorders. The use of heparin, however, may cause haemorrhage as a side effect.

Heparin has been gradually replaced by low-molecular-weight heparins which cause less undesirable side effects. These low-molecular-weight heparins are prepared by fractionation, controlled depolymerization of heparin or by chemical synthesis. Low molecular weight heparin is currently utilized clinically as an anticoagulant for a wide spectrum of pathogenic conditions, particularly in the management of acute coronary ischemic syndromes and venous thromboembolism events (2).

Low molecular weight heparin activates the protease inhibitor antithrombin, thereby resulting in inhibition of serine proteases (primarily thrombin and Factor Xa) in the coagulation cascade. It is unexpected that low molecular weight heparin can be used to prevent and treat sepsis caused by S. aureus. The protective effects of low molecular weight heparin against sepsis may be directly or indirectly related to any or all of the following modes of actions:
a) inhibition of factor Xa and IIa activities;
b) direct binding to bacteria and prevention microbial attachment and colonization;
c) attenuation of hyper-inflammatory cascade events associated with systemic inflammatory response syndrome;
d) reduction or prevention of disseminated intravascular coagulation, a frequent prologue in septic death;
e) amelioration of organ hypoperfusion and fluid-refractory hypotension, critical features of sepsis and septic death.

According to the invention, a low molecular weight heparin having an average molecular weight of between 1000 and 10,000 daltons, in particular between 1500 and 6000 daltons, and in particular between 4000 and 5000 daltons is preferably used.

The pharmacokinetics of low molecular weight heparin is generally known in the art. Low molecular weight heparins produce a more predictable anticoagulant response than unfractionated heparin due to their better bioavailability, longer half-life, and dose independent clearance. The plasma half-life of low molecular weight heparin is 2-4 times as long as that of unfractionated heparin (2-4 hrs after intravenous injection and 3-6 hrs after subcutaneous injection). The pharmacokinetic differences between low molecular weight heparin and unfractionated heparin is explained by the decreased binding of low molecular weight heparin to plasma proteins, endothelial cells and macrophages.

After years of intensive basic and clinical research, low molecular weight heparins have been clearly established as efficacious in several clinical settings, including treatment and prevention of venous thromboembolic disease, treatment of unstable coronary ischemic disease, and treatment of acute cerebrovascular ischemia. Low molecular weight heparins have also been proven to be at least as effective as intravenous unfractionated heparin in the treatment of unstable angina.

Thus, in view of the current clinical experiences with low molecular weight heparins, one of ordinary skill in the art could readily determine the appropriate route of administration and dosage according to the age, weight and any other factors specific to the subject to be treated. The doses usually depend on the desired effect, the duration of treatment and the route of administration used.

The present invention in one aspect or embodiment presents the use of the glycosaminoglycans, chondroitin sulfate A and dermatan sulfate in the treatment of S.aureus-induced sepsis. Both these compounds have been shown to possess anticoagulative and antithrombotic properties.

In yet another embodiment of the invention is a correlation between different doses of low molecular weight heparins or chondroitin sulfate A or dermatan sulfate and response in the treatment of S. aureus-induced sepsis in mice. For example, it is shown that doses lower than 0.5 mg/kg body weight per mouse per day was better for survival of S. aureus-induced septic mice as compared to doses greater than 1 mg/kg body weight per mouse per day (FIG. 2).

The invention also presents a partial elucidation of the mechanism of action in dermatan sulfate protection in S. aureus-induced sepsis. To ascertain the physiological pathway that is modulated by dermatan sulfate, the coagulation profiles of mice treated with low and high concentartions of dermatan sulfate in the context of S.aureus-induced sepsis was assessed. High doses of dermatan sulfate in context of the sepsis was found to increase the prothrombin time. On the other hand low doses of dermatan sulfate was found to decrease the partial throboplastin time in septic mice.

This invention also evinces that dermatan sulfate has the capacity to modulate levels of plasma protein C levels. Low doses of dermatan sulfate (<10 mg/Kg) appear to decrease protein C levels in the context of sepsis. Conversely, high levels of dermatan sulfate (>20 mg/kg) appear to increase plasma C levels after the induction of sepsis.

The beneficial effects of dermatan sulfate may be directly or indirectly attributable to one or all of the following: 1) Stabilization of plasma protein C levels, 2) Enhancement of activated protein C (APC) activity, 3) Activation of heparin Cofactor-II, an extravascular inhibitor of thrombin, and 4) Replenishment of depleted source of dermatan sulfate from host proteoglycans such as decorin, thrombomodulin, versican, biglycan, endocan and epiphycan.

The invention also presents that the pivotal coagulation factors being affected in early sepsis include factors VIII, IX, XI, XII, High molecular Weight Kininogen (HMWK) and pre-kallikren. This was deduced on the basis of a contracted prothrombin time and prolonged partial thromboplastin time seen in mice with S. aureus-induced sepsis.

The medicaments of the present invention may comprise a salt (preferably sodium or calcium) of a low-molecular weight heparin or chondroitin sulfate A or dermatan sulfate in combination with any other pharmaceutically compatible product that may be inert or physiologically active. The medicaments may be administered via the intravenous, intraperitoneal, subcutaneous, or topical route.

Sterile pharmaceutical compositions for intravenous or subcutaneous administration are generally aqueous solutions. These compositions may also contain wetting, isotonizing, emulsifying, dispersing and/or stabilizing agents. Sterilization can be carried out in several ways, for example by aseptisizing filtration, by incorporating sterilizing agents into the composition, or by irradiation.

A number of low molecular weight heparins are known in the art, and they are suitable for use according to the present invention. These include enoxaparin marketed by Rhone-Poulenc Rorer, nadroparin marketed by Sanofi, parnaparin marketed by Opocrin-Alfa, reviparin marketed by Knoll, dalteparin marketed by Kabi Pharmacia, tinzaparin marketed by Novo Nordisk, danaparoid marketed by Organon, ardeparin developed by Wyeth Ayerst, certoparin marketed by Sandoz and products under study such as CY222 from Sanofi-Choay (Thromb. Haemostasis, 58:553 (1987)), and SR90107/ORG31540 from Sanofi-Organon (Thrombosis and Haemostasis, 74:1468-1473 (1995)).

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Use of Low Molecular Weight Heparin in the Prevention and Treatment of Sepsis

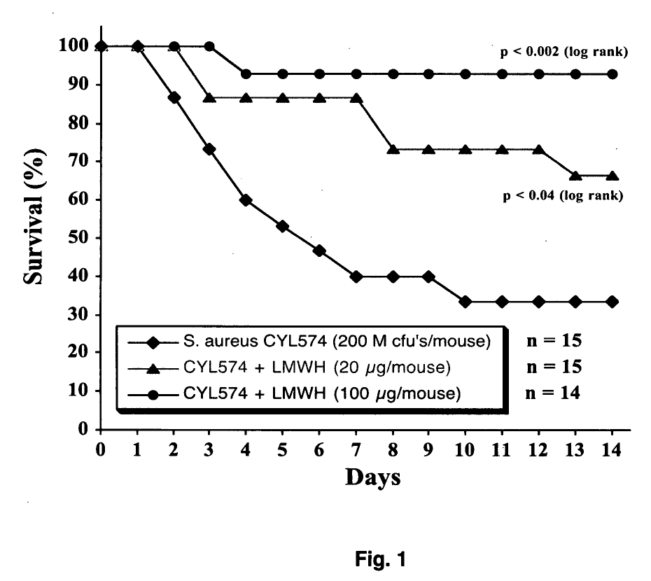

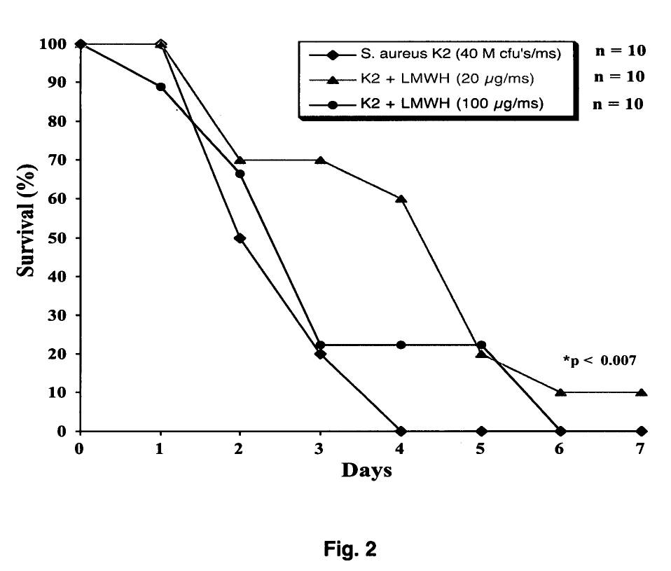

Experiments evaluating the effects of low molecular weight heparin in preventing mortality and/or prolonging survival were conducted in an animal model of S. aureus-induced septic death. FIG. 1 shows the effects of low molecular weight heparin after inception of sepsis. Mice that were treated with a prophylaxis dose (1 mg/kg) of low molecular weight heparin exhibited a survival rate of 66.7% 14 days after infection as compared to 33.3% in the control group treated with PBS. Mice treated with a high dose (5 mg/kg) of low molecular weight heparin exhibited a survival rate of 92.8% after infection (p=0.0017 versus control).

FIG. 2 shows the effects of low molecular weight heparin after infection with a supra-lethal dose of the highly pathogenic S. aureus clinical isolate K2. Mice treated with a prophylaxis dose (1 mg/kg) of low molecular weight heparin had a survival rate of 70% versus 20% in the PBS control group at 72 hr, and survival rate of 60% versus 0% in the control group at 96 hr (p=0.01 versus control). Mice treated with high doses (5 mg/kg) of low molecular weight heparin exhibited a survival rate of 22.2% as compared to 0% in the control group 96 hours after infection.

The therapeutic index of low molecular weight heparin was also evaluated by using several-fold concentrations in excess of the clinical dose (1 mg/kg or 20 μg/mouse). When doses of 100 or 50 μg of low molecular weight heparin per mouse were utilized lower survival rates were observed as compared to control mice treated only with PBS (data not shown). It is noteworthy to emphasize that many of the mice in the low molecular weight heparin group exhibited signs of bleeding after only 48 hr. These adverse effects included hematuria, subcutaneous hematomas, and bleeding at the site of injection. The majority of the mice that died in the low molecular weight heparin group were documented with at least one episode of bleeding. These observations suggested that mice treated with high doses of the low molecular weight heparin group were either excessively anticoagulated or experienced the effects of disseminated intravascular coagulation. Insight into the actual cause of bleeding can be gained by examining the coagulation profiles.

### EXAMPLE 2

Dose Response Study with LMWH

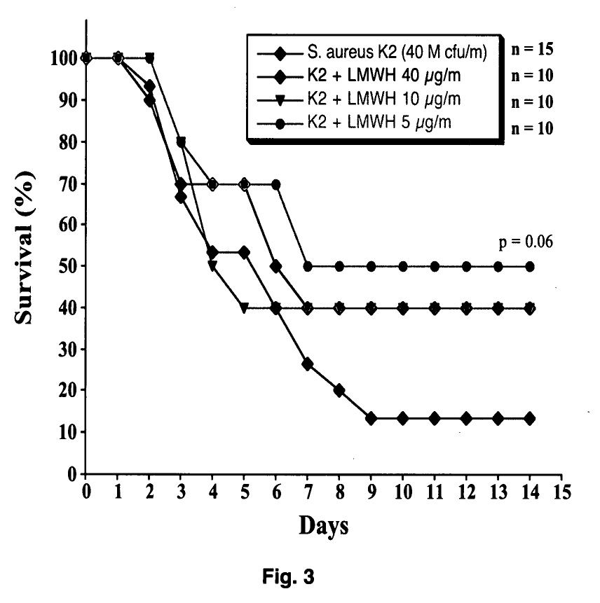

A dose response study with low molecular weight heparin was carried out in an animal model of S. aureus-induced sepsis. Infected mice were injected subcutaneously with increasing doses of low molecular weight heparin ranging from 540 μg at 2 hours and subsequently every twenty four hours (FIG. 3).

It is clear from FIG. 3 that doses approximately <0.5 mg of low molecular weight heparin/kg per mouse per day confers increased survival as compared to infected mice dosed with >1 mg of low molecular weight heparin/kg body weight per mouse per day. This result also suggests that low molecular weight heparin has a narrow therapeutic window.

### EXAMPLE 3

Effects of Chondroitin Sulfate A in S.aureus-induced Sepsis

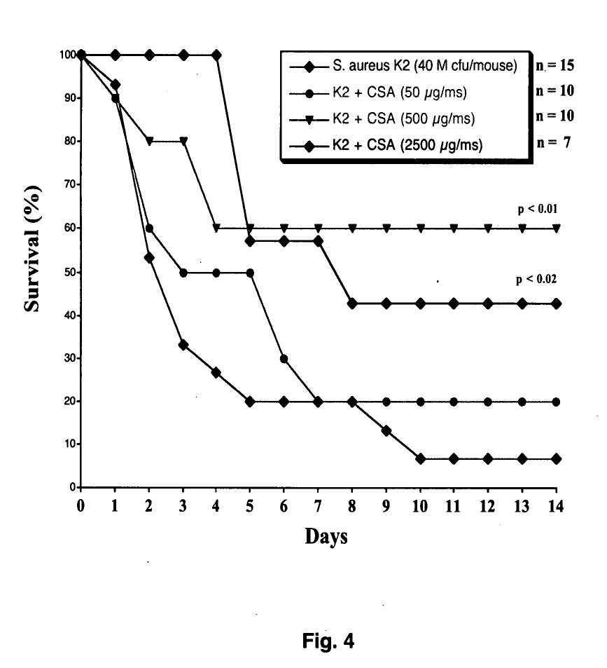

Chondroitin sulfate A (CSA), a glycosaminoglycan, was tested to evaluate its therapeutic effect (FIG. 4) in an animal model of S. aureus-induced sepsis. Infected mice were treated to chondroitin sulfate A in a dose range of 50-2500 mg of chondroitin sulfate A at 2 hours and subsequently every twenty four hours.

FIG. 4 clearly shows that chondroitin sulfate A, when injected at a high dose of greater than 10 mg/kg body weight or greater than 200 μg per mouse have prolonged survival as compared to infected mice treated with PBS. It was further seen that very high doses (>100 mg/kg body weight) of chondroitin sulfate A confer augmented survival during the first days after the onset of sepsis. However, continued daily high doses of chondroitin sulfate A appear to be detrimental in infected mice.

### EXAMPLE 4

Use of Dermatan Sulfate in the Prevention and Treatment of Sepsis

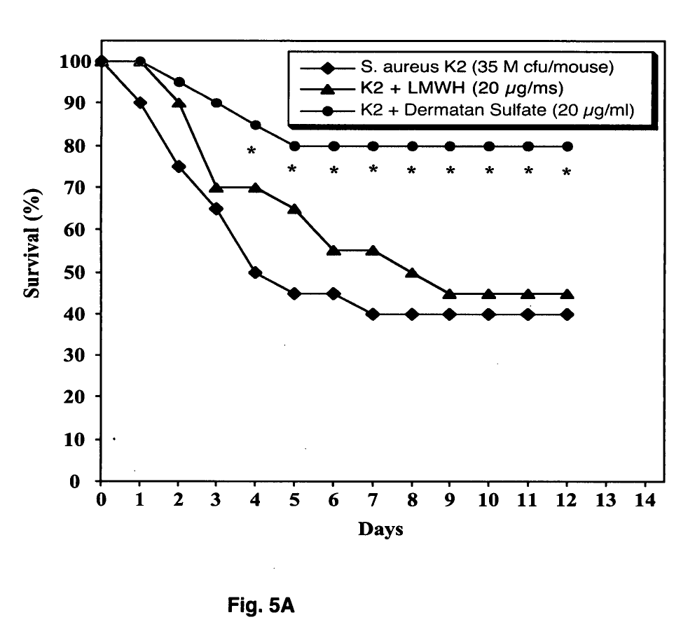

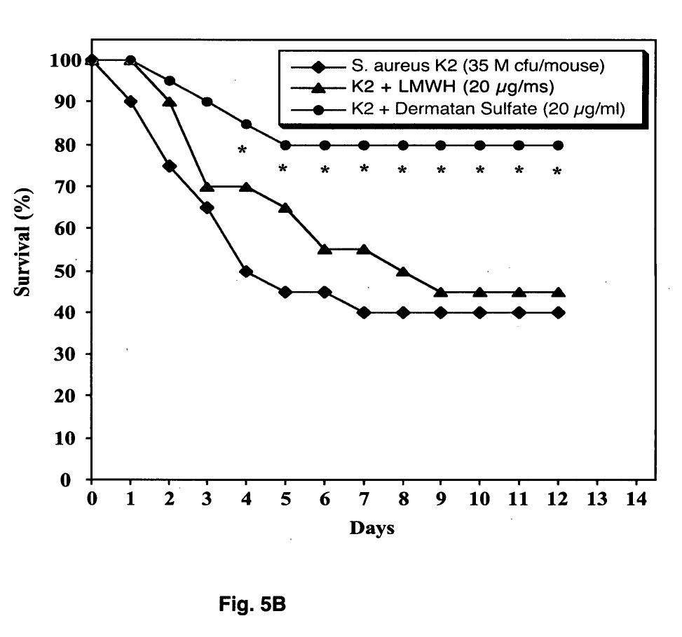

The biochemical specificity of low molecular weight heparin was evaluated by direct comparison with another biologically relevant glycosaminoglycan. FIGS. 5A and 5B show the effect of both low molecular weight heparin and dermatan sulfate on S. aureus-induced death. Mice that were treated subcutaneously with low molecular weight heparin exhibited a higher rate a survival than the control group injected with PBS. Interestingly, the mice treated with dermatan sulfate also displayed a substantial survival rate. The glycosaminoglycan groups also displayed a healthier clinical profile with less ruffled fur and higher alertness and activity.

A second experiment with a larger group of animals was subsequently conducted. Again, the low molecular weight heparin-treated mice exhibited a higher survival rate than the PBS-treated mice. Moreover, the dermatan sulfate-treated mice displayed a dramatic survival rate as compared to both the low molecular weight heparin and control groups.

### EXAMPLE 5

Dose Response Study with Dermatan Sulfate

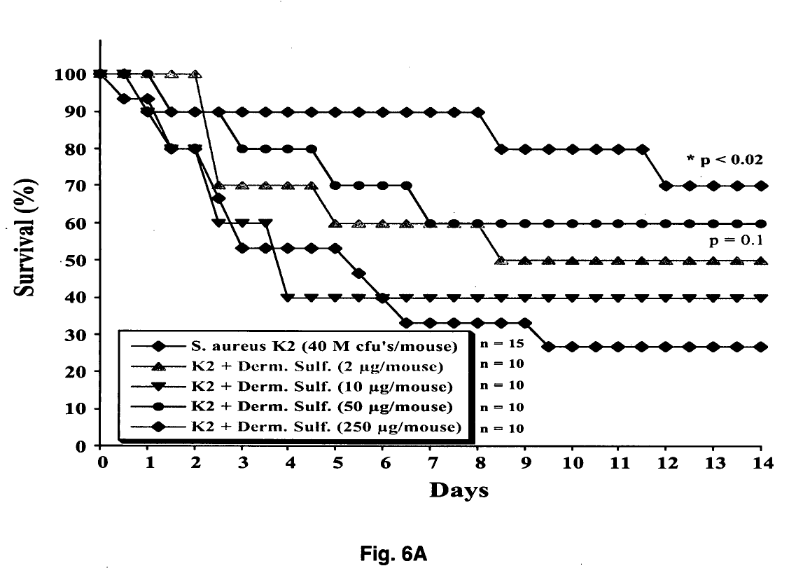

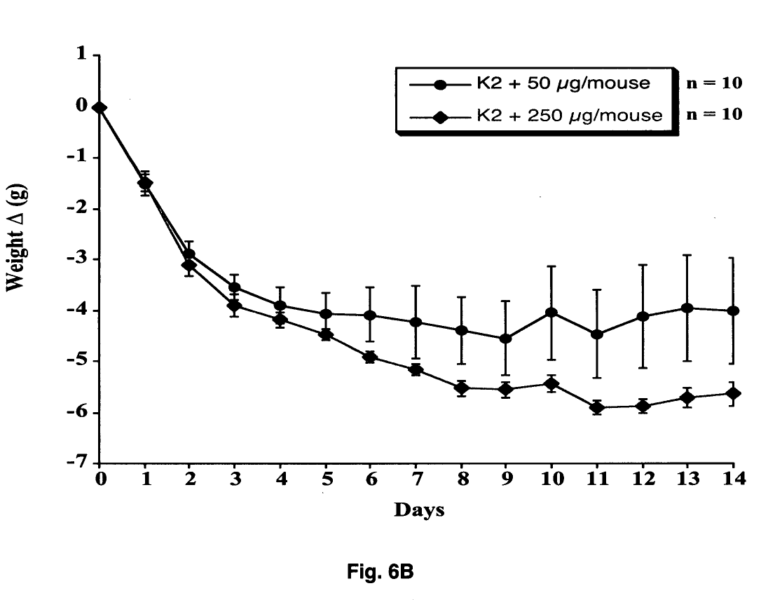

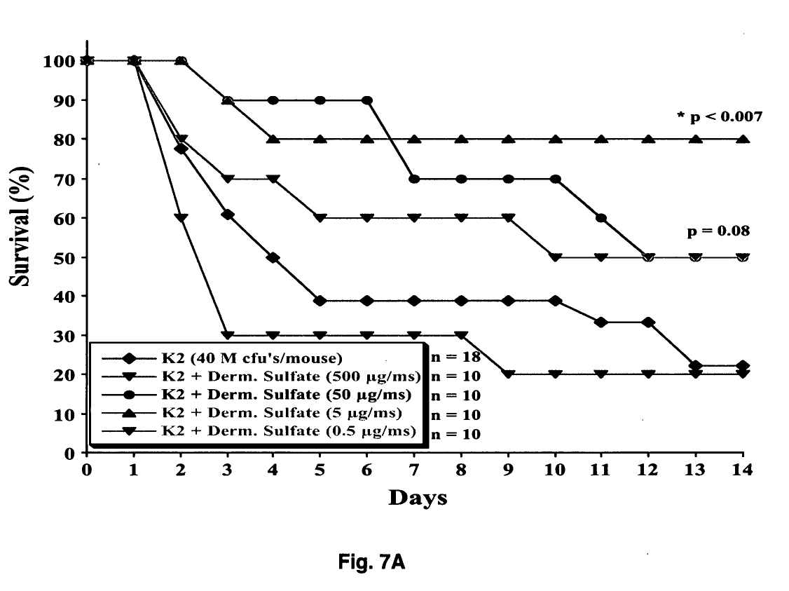

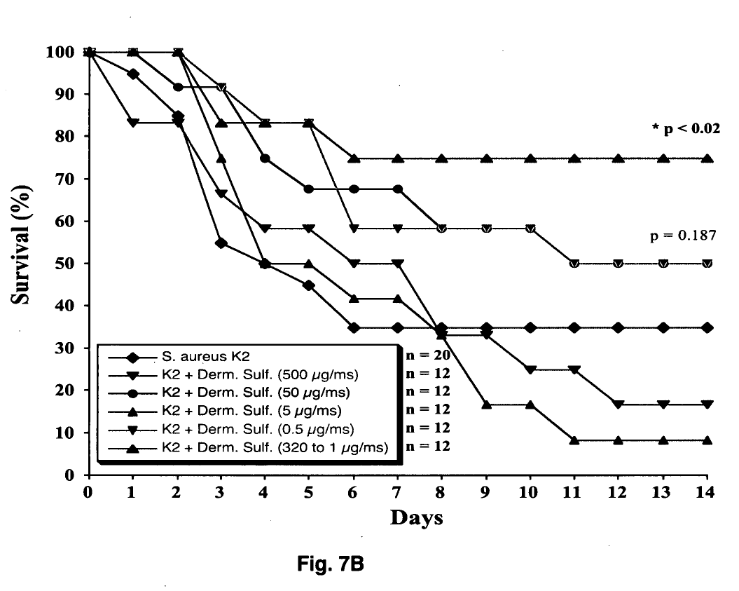

A dose response study with dermatan sulfate (DS) was carried out in an animal model of S. aureus-induced sepsis. Infected mice were injected subcutaneously with increasing doses of dermatan sulfate ranging from 0.1-25 mg/Kg body weight or 2-500 μg at two hours and subsequently every twenty four hours (FIGS. 6A, 6B, 7A and 7B). These studies revealed that high doses (>1 mg/Kg) appear to be more protective than lower doses (<1 mg/Kg) of dermatan sulfate. When the weight of the surviving animals was analyzed, mice exhibiting a higher survival rate substantially lost more weight than mice treated with the lower doses (FIG. 6B). This result suggests that dermatan sulfate is not exerting its effects directly on the pathogen but coversely, modulating a host pathway that results in the delay of organ dysfunction and failure.

A very high dose of approximately 25 mg/kg body weight is detrimental to the infected mice after the onset of sepsis (FIG. 7A). Mice injected with 500 μg/day of dermatan sulfate did clinically well during the first twenty four to forty eight hours. However, they rapidly declined and subsequently showed poor survival. But, a high dose of dermatan sulfate was non-toxic to non infected control mice treated with 500 μg/day of dermatan sulfate.

To overcome high dose toxicity in infected mice, a tapered dose experiment was conducted (FIG. 7B). A group of infected mice were treated with a tapering dose of dermatan sulfate beginning at 16 mg/kg body weight and decreased by half every day until the last dose on day 13 of approximately 0.025 mg/Kg body weight was reached. FIG. 7B clearly shows that a tapered dose of dermatan sulfate yields optimal survival rates in the treated mice.

### EXAMPLE 6

Effect of Dermatan Sulfate on the Extrinsic Pathway of Coagulation in S. aureus-induced Sepsis

To ascertain the physiological pathway that is modulated by dermatan sulfate, coagulation profiles of mice treated with low and high concentrations of the compound in the context of S. aureus-induced sepsis was assessed. The extrinsic pathway of coagulation was first evaluated by measuring the prothrombin time (PT) in the plasma of mice forty eight hours after infection with S. aureus and treatment with dermatan sulfate. K2 was grown to log phase and set via a nephelometer to a lethal concentration of 40 million cfu per mouse. Balb/c mice were infected intravenously with 40 m cfu of S. aureus K2 on day 0. Treatment groups were injected subcutaneously with increasing doses of dermatan sulfate (50 or 500 μg dermatan sulfate per mouse) at thirty minutes and subsequently every twenty four hours. Control group mice were injected subcutaneously with PBS or dermatan sulfate (50 or 500 μg dermatan sulfate per mouse per day). Mice were bled at 48 hours after infection via tail vein. Blood was collected in 0.12 M sodium citrate in a 9:1 ratio of blood to citrate. Samples were centrifuged and plasma collected and frozen at −20° C. until use. Plasmas were diluted 1:3 in Owrens buffer for analysis. Prothrombin time (PT) was determined utilizing an XM coagulometer according to the manufacturer's instructions.

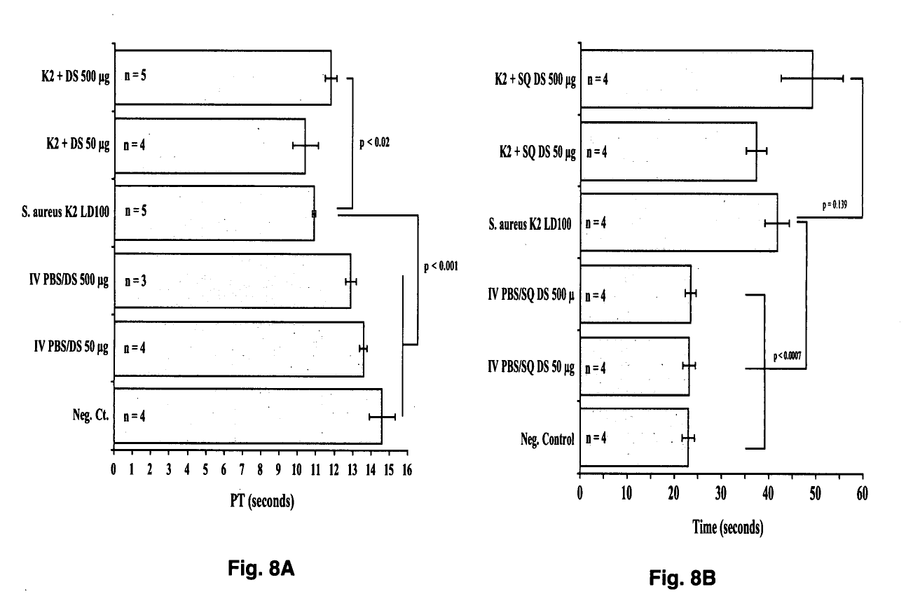

FIG. 8A shows that mice that were infected with a LD_{80-100%} of S. aureus and treated with PBS exhibited a contracted prothrombin time. This indicates that one or more factors in this pathway are increased in septic mice. Infected mice treated with dermatan sulfate also showed contracted prothrombin times. There was a significant increase in prothrombin time in infected mice treated with 500 μg/day of dermatan sulfate as compared to infected PBS treated mice.

### EXAMPLE 8

Effect of Dermatan Sulfate on the Extrinsic Pathway of Coagulation in S. aureus -induced Sepsis

The effect of dermatan sulfate on the intrinsic pathway of coagulation was determined by measuring the active partial thromboplastin time. Plasma samples from S.aureus infected mice was prepared as described in example 7. Partial thromboplastin time (PTT) was determined utilizing an XM coagulometer according to the manufacturer's instructions. FIG. 8B shows that mice infected with a LD_{80-100%} of S.aureus and treated with PBS exhibited a dramatically prolonged partial thromboplastin time time as compared to control animals. This indicates that one or more factors in the intrinsic pathway of coagulation are significantly decreased or depleted in PBS treated septic mice. Infected mice treated with dermatan sulfate also showed prolonged partial thromboplastin time times. There was a slight decrease of the partial thromboplastin time time in infected mice treated with a daily dose of 50 μg/day of dermatan sulfate as compared to infected PBS treated mice. This suggests that daily low doses of dermatan sulfate may contribute to the stabilization of hemostasis and thus confer a survival advantage after the onset of sepsis. FIG. 8B also shows that septic mice treated with 500 μg/day of dermatan sulfate exhibit significantly prolonged partial thromboplastin time time as compared to PBS treated infected mice.

### EXAMPLE 9

Coagulation Factors Affected Early in S. aureus-induced Sepsis

Plasma samples from S.aureus infected mice were prepared as described in Example 7. Prothrombin time and partial thromboplastin time (PTT) were determined utilizing an XM coagulometer according to the manufacturer's instructions.

FIGS. 8A and 8B show that septic mice have contracted PT time and prolonged partial thromboplastin time time. These results suggest that the pivotal coagulation factors being affected early in sepsis include Factors VIII, IX, Xl, XII, High Molecular Weight Kininogen and Pre-kallikrein.

### EXAMPLE 10

Fibrinogen Levels of Septic Mice Treated with Dermatan Sulfate

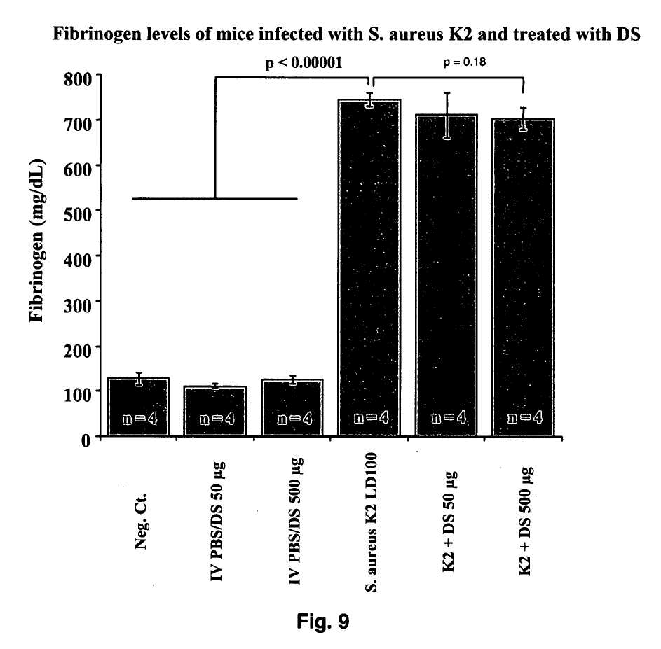

Plasma samples from S.aureus infected mice were prepared as described in example 7. Fibrinogen levels were determined utilizing control standards using an XM coagulometer according to manufacturer's instructions. It was seen that mice infected with a LD_{80-100%} of S.aureus and treated with PBS exhibited significantly high levels of fibrinogen (FIG. 9). Infected mice treated with 50-500 μg/day of dermatan sulfate did not exhibit any significant difference in fibrinogen levels as compared to the infected PBS treated mice.

### EXAMPLE 11

Protein C Levels of Septic Mice Treated with Dermatan Sulfate

Plasma samples from S.aureus infected mice were prepared as described in Example 7. Protein C in the plasma was determined as percent levels based on standards and their ability to prolong partial thromboplastin time. Clotting assay was evaluated using an XM coagulometer according to manufacturer's instructions.

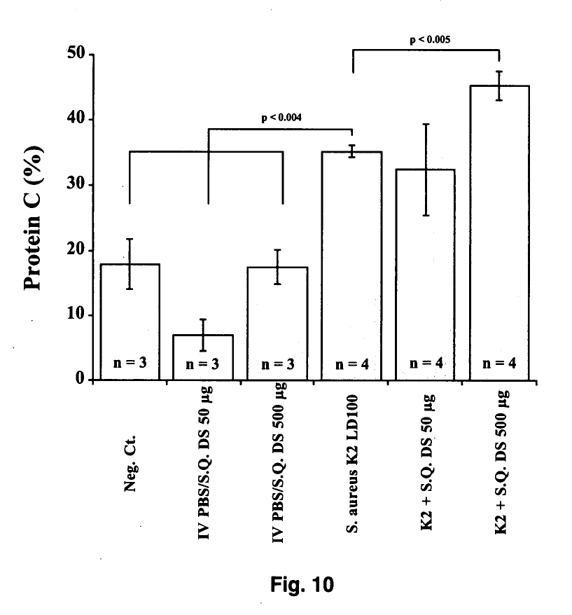

It was seen that mice infected with a LD_{80-100%} of S.aureus and treated with PBS exhibited significantly high levels of protein C (FIG. 10). Infected mice treated with 50 μg/day of dermatan sulfate exhibited a broad range of protein c levels that were higher and lower than infected mice treated with PBS. This suggests that low doses of dermatan sulfate may have the capacity to reduce or normalize) plasma protein C levels in the context of sepsis and thus yield a beneficial survival rate. Conversely, 500 μg/day of dermatan sulfate appear to dramatically increase protein C levels which may induce rapid depletion of protein C and yield poor survival.

### SUMMARY

The present invention discloses a method of treating sepsis or a related disorder caused by bacterial infection in an animal, comprising the step of administering a therapeutically effective amount of a glycosoaminoglycan to the subject. The glycosaminoglycan can be low molecular weight heparin, dermatan sulfate, chondroitin sulfate A, chondroitin sulfate C and heparan sulfate and the subject can be a human or a non-human animal. The bacterial infection can be caused by gram-positive (Enterococcus spp., Staphylococcus spp., and Streptococcus spp.) or gram-negative bacteria.

The present invention in one embodiment provides a method of treating sepsis or a related disorder caused by bacterial infection with a gycosaminoglycan where the bacteria causing the infection is resistant to one or more antibiotics. Furthermore, this glycosaminoglycan can be administered by subcutaneous injection, intraperitoneal injection or intravenous injection.

The present invention in one embodiment gives a method of treating sepsis or a related disorder caused by a bacterial infection with a glycosaminoglycan in conjunction with an antibiotic.

The invention further discloses that the molecular weight of low molecular weight heparin administered for the treatment of sepsis or a related disorder caused by bacterial infection can range from 1000-10,000 daltons. This low molecular weight heparin can be enoxaparin, nadroparin, parnaparin, reviparin, dalteparin, tinzaparin, danaparoid, ardeparin, certoparin, and products under study such as CY222 and SR90107/ORG31540

The invention in one embodiment discloses that the glycosaminoglycan can be administered in a dose range of 0.5-25 mg/kg body weight of the animal. The invention further provides a method of tapering the dose where the infected animal is started on a high dose of the glycosaminoglycan and then the dose is slowly reduced over the treatment period to overcome high dose toxicity.

The invention also discloses a method of treating sepsis or related disorder caused by a bacterial infection in an animal by administering a pharmacologically effective dose of a glycosaminoglycan wherein the glycosaminoglycan stabilizes the prothrombin time or thromboplastin time in said animal.

The invention further discloses a method of treating sepsis or related disorder caused by a bacterial infection in an animal by administering a pharmacologically effective dose of a glycosaminoglycan wherein the glycosaminoglycan stabilizes protein C levels in the plasma of said animal. The following references were cited herein:
1. Riedemann N C, Guo R F, and Ward P A: Novel strategies for the treatment of sepsis. Nat.Med. (2003) 9:517-24.
2. Hirish. J, Dalen J, and Guyatt D: The sixth (2000) ACCP guidelines for antithrombic therapy for prevention and treatment of thrombosis. Chest (2001) 119:1S-2S.

Any publications mentioned in this specification are indicative of the levels of those skilled in the art to which the invention pertains. Further, these publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method of treating sepsis or a related disorder caused by bacterial infection in an animal, comprising the step of administering a therapeutically effective amount of a glycosoaminoglycan to said subject.

2. The method of claim 1, wherein said glycosoaminoglycan is low molecular weight heparin, dermatan sulfate, chondroitin sulfate A, chondroitin sulfate C and heparan sulfate.

3. The method of claim 1, wherein said subject is a human or a non-human animal.

4. The method of claim 1, wherein said bacterial infection is caused by gram-positive or gram-negative bacteria.

5. The method of claim 4, wherein said bacteria are resistant to one or more antibiotics.

6. The method of claim 4, wherein said gram-positive bacteria are selected from the group consisting of Enterococcus spp., Staphylococcus spp., and Streptococcus spp.

7. The method of claim 6, wherein said Staphylococcus bacteria is Staphylococcal aureus.

8. The method of claim 1, wherein glycosoaminoglycan is administered by a method selected from the group consisting of subcutaneous injection, intraperitoneal injection, and intravenous injection.

9. The method of claim 2, wherein said low molecular weight heparin has an average molecular weight of between 1000 and 10,000 daltons.

10. The method of claim 2, wherein said low molecular weight heparin has an average molecular weight of between 1500 and 6000 daltons.

11. The method of claim 2, wherein said low molecular weight heparin has an average molecular weight of between 4000 and 5000 daltons.

12. The method of claim 2, wherein the low molecular weight heparin is enoxaparin.

13. The method of claim 2, wherein the low molecular weight heparin is nadroparin.

14. The method of claim 2, wherein the low molecular weight heparin is parnaparin.

15. The method of claim 2, wherein the low molecular weight heparin is reviparin.

16. The method of claim 2, wherein the low molecular weight heparin is dalteparin.

17. The method of claim 2, wherein the low molecular weight heparin is tinzaparin.

18. The method of claim 2, wherein the low molecular weight heparin is danaparoid.

19. The method of claim 2, wherein the low molecular weight heparin is ardeparin.

20. The method of claim 2, wherein the low molecular weight heparin is certoparin.

21. The method of claim 2, wherein the low molecular weight heparin is CY222.

22. The method of claim 2, wherein the low molecular weight heparin is SR90107/ORG31540.

23. The method of claim 1, further comprises the step of administering to said animal an effective amount of an agent that treats said bacterial infection.

24. The method of claim 23, wherein said agent is an antibiotic.

25. The method of claim 1, wherein said glycosaminoglycan is administered in a dose range of 0.5-25 mg/kg body weight of said animal.

26. The method of claim 2, wherein said glycosaminoglycan is administered in a tapered dose such that the dose on day one of the treatment is approximately 25 mg/Kg body weight of the animal and the dose is subsequently decreased on each day of the treatment wherein the dose on the final day of treatment is approximately 0.5 mg/Kg body weight of the animal.

27. A method of treating sepsis or a related disorder caused by bacterial infection in an animal, by administering a pharmacologically effective dose of a glycosaminoglycan wherein said compound stabilizes the prothrombin time in said subject.

28. The method of claim 27, wherein said glycosaminoglycan is low molecular weight heparin, dermatan sulfate, chondroitin sulfate A, chondroitin sulfate C and heparan sulfate.

29. A method of treating sepsis or a related disorder caused by bacterial infection in an animal, by administering a pharmacologically effective dose of a glycosaminoglycan wherein said compound stabilizes the partial thromboplastin time in said subject.

30. The method of claim 29, wherein said glycosaminoglycan is low molecular weight heparin, dermatan sulfate, chondroitin sulfate A, chondroitin sulfate C and heparan sulfate.

31. A method of treating sepsis or a related disorder caused by bacterial infection in an animal, by administering a pharmacologically effective dose of a glycosaminoglycan wherein said compound stabilizes the levels of protein C in the plasma of said subject.

32. The method of claim 31, wherein said glycosaminoglycan is low molecular weight heparin, dermatan sulfate, chondroitin sulfate A, chondroitin sulfate C and heparan sulfate.
